(19) United States
(12) Patent Application Publication
(10) Pub. No.: US 2026/0153461 A1
(43) Pub. Date: Jun. 4, 2026

(54) SYSTEM AND METHOD FOR MEASURING A QUANTITY OF A KNOWN GAS

(71) Applicant: Inficon Inc., East Syracuse, NY (US)

(72) Inventor: Shawn Wilson, Liverpool, NY (US)

(21) Appl. No.: 19/400,012

(22) Filed: Nov. 25, 2025

Related U.S. Application Data
(60) Provisional application No. 63/728,047, filed on Dec. 4, 2024.

(51) Int. Cl. G01N 25/18; G01N 33/00

(52) U.S. Cl. CPC G01N 25/18; G01N 33/0006; G01N 33/0016; G01N 33/0022

(57) ABSTRACT

A system and method for continuously monitoring and measuring a quantity of a reference gas includes providing a reference gas and measuring a thermal conductivity of the reference gas via a gas analyzer and calculating a purity threshold value based on the measured thermal conductivity of the reference gas. A mixture of gases containing the reference gas is then provided and a thermal conductivity of the mixture of gases is continuously measured via the gas analyzer. The measured thermal conductivity of the mixture of gases is compared to the purity threshold value, and a purity threshold level of the reference gas in the mixture of gases is indicated once the measured thermal conductivity of the mixture of gases reaches the purity threshold value.

### FIELD OF THE INVENTION

The present technology relates generally to the field of gas sensors. More particularly, the present technology relates to a system and method of measuring and signaling an amount of a known gas, such as hydrogen.

### BACKGROUND OF THE INVENTION

The field of gas sensing encompasses a broad range of technologies aimed at detecting and quantifying specific gaseous species in diverse environments. With advances in sensor materials, electronics, and data processing, these systems have evolved to deliver increasingly precise and reliable measurements. Among the various target gases, hydrogen has garnered particular attention due to its growing use in energy, manufacturing, and transportation sectors. Monitoring gas composition in real time not only supports process control and quality assurance but also plays a significant role in safety management, especially when highly flammable or reactive gases are involved. As production methods and applications expand, the demand for robust monitoring systems that can adapt to different process streams, pressure ranges, and environmental conditions continues to intensify.

Hydrogen purging has become a cornerstone technique in many industrial workflows to establish reducing or inert atmospheres. In metal annealing and heat treatment, purging prevents oxidation and surface degradation, enhancing final mechanical properties. Semiconductor fabs rely on hydrogen to eliminate moisture and oxygen prior to high-temperature processes, safeguarding device performance. In the petrochemical and chemical industries, hydrotreating and hydrogenation reactions hinge on tight control of hydrogen levels. Likewise, pharmaceutical syntheses, glass manufacturing, and fuel-cell production all leverage hydrogen purging to secure product purity and prevent contamination. Automotive applications – most notably hydrogen fuel cell vehicles – depend on accurate monitoring to ensure component integrity and operational safety. Across these sectors, both the efficacy of the purge and the downstream quality metrics hinge on reliable gas concentration data.

While these methods can achieve acceptable accuracy, they tend to rely on sophisticated instrumentation, periodic calibration routines, and complex fluidic arrangements. The associated capital costs, maintenance demands, and operational overhead can be prohibitive, particularly in applications that call for compact, continuously operating units with minimal intervention.

The existing gas measurement systems and methods typically look for and measure concentrations of impurities and subtract those to indicate a hydrogen concentration. For example, U.S. Patent No. 3,836,449 discusses the use of an initial flow of pure hydrogen carrier gas as a first reference signal, then introduces impurities to provide a second signal of the quantity of impurities.

Another hydrogen measuring system is described in U.S. Patent No. 4,226,675, which discusses measuring hydrogen in a reference filament for baseline, then modifying the constituency of the same gas sample and cycling it through the measuring filament. U.S. Patent No. 1,860,544 discusses a thermal conductivity gas analysis apparatus that uses a standard reference cell filled with a liquid or solid, and a measurement cell filled with the gas to be analyzed.

While these methods can achieve acceptable accuracy, they tend to rely on sophisticated instrumentation, periodic calibration routines, and complex fluidic arrangements. The associated capital costs, maintenance demands, and operational overhead can be prohibitive, particularly in applications that call for compact, continuously operating units with minimal intervention.

In many purging operations, accurately identifying the moment when the target gas concentration crosses a predetermined threshold is important, rather than merely assessing impurity levels. Delayed response, calibration drift, or difficulty in distinguishing between similar conductivity changes can result in prolonged purge cycles, decreased throughput, and potential safety concerns. Additionally, large analyzers and high-precision auxiliary equipment increase complexity and restrict deployment in space-limited or portable environments. An approach is required that can directly indicate the achievement of a reference gas purity level in real time, utilizing simplified hardware and control logic. Such a method would decrease system complexity, reduce ownership costs, and improve responsiveness in manufacturing and energy-related processes.

What is needed, therefore, is an improved system and method of measuring and signaling a reference gas, e.g., hydrogen gas, concentration that address at least the problems described above.

### SUMMARY OF THE INVENTION

According to an example embodiment of the present invention, a system for continuously monitoring and measuring a quantity of a reference gas is provided, including a reference gas inlet fluidly coupled to a reference gas conduit, a sample gas inlet fluidly coupled to a sample gas conduit, a gas analyzer fluidly coupled to the reference gas conduit and the sample gas conduit and comprising a sample gas flow channel and a reference gas flow channel, wherein the reference gas conduit is fluidly coupled to both the reference gas flow channel and the sample gas flow channel, and a controller connected to the gas analyzer. The gas analyzer is configured to measure a thermal conductivity of the reference gas and a thermal conductivity of a sample gas containing the reference gas and at least one other gas. The controller is configured to calculate a purity threshold value based on the thermal conductivity of the reference gas and to compare the thermal conductivity of the sample gas to the purity threshold value to indicate a purity threshold level of the reference gas in the sample gas once the thermal conductivity of the sample gas reaches the purity threshold value.

In some embodiments, the gas analyzer is a thermal conductivity detector.

In some embodiments, the gas is hydrogen gas.

In certain embodiments, a first valve positioned in the reference gas conduit and configured to control flow of the reference gas to the sample gas flow channel, and a second valve positioned in the sample gas conduit and configured to control flow of the sample gas to the sample gas flow channel. The controller is configured to open the first valve to allow flow of the reference gas to the sample gas flow channel to calculate the purity threshold value, and to open the second valve to allow flow of the sample gas to the sample gas flow channel to measure the thermal conductivity of the sample gas.

In some embodiments, a first flow meter positioned in the reference gas conduit downstream from the reference gas inlet and configured to measure a flow rate of the reference gas, and a second flow meter positioned in the sample gas conduit downstream from the sample gas inlet and configured to measure a flow rate of the sample gas. In certain of these embodiments, a first restrictor positioned in the reference gas conduit downstream from the first flow meter and configured to adjust at least one of the flow rate and a pressure of the reference gas, and a second restrictor positioned in the sample gas conduit downstream from the second flow meter and configured to adjust at least one of the flow rate and a pressure of the sample gas.

The present technology also provides a method for continuously monitoring and measuring a quantity of a reference gas. The method includes providing a reference gas and measuring a thermal conductivity of the reference gas via a gas analyzer, calculating a purity threshold value based on the measured thermal conductivity of the reference gas, providing a mixture of gases containing the reference gas and continuously measuring a thermal conductivity of the mixture of gases via the gas analyzer, comparing the measured thermal conductivity of the mixture of gases to the purity threshold value, and indicating a purity threshold level of the reference gas in the mixture of gases once the measured thermal conductivity of the mixture of gases reaches the purity threshold value.

The reference gas may be hydrogen gas.

In some embodiments, the method also includes the step of calibrating the gas analyzer based on the measured thermal conductivity of the substantially pure reference gas.

In certain embodiments, the substantially pure reference gas is continuously provided to the gas analyzer and the thermal conductivity of the substantially pure reference gas is continuously measured, and the purity threshold value is continuously updated to remove changing environment variables.

In some embodiments, the method also includes measuring a flow rate of at least one of the substantially pure reference gas and the mixture of gases, and adjusting the flow rate of substantially pure reference gas and the flow rate of the mixture of gases to be substantially equal.

In another embodiment of the present technology, a method for detecting a quantity of hydrogen gas during a purge of at least one other gas with the hydrogen gas is provided. The method includes providing a substantially pure hydrogen gas and measuring a thermal conductivity of the substantially pure hydrogen gas via a thermal conductivity detector, calculating a purity threshold value based on the measured thermal conductivity of the substantially pure hydrogen gas, supplying the substantially pure hydrogen gas to a vessel containing the at least one gas, providing a mixture of gases from the vessel to the thermal conductivity detector and measuring a thermal conductivity of the mixture of gases, comparing the measured thermal conductivity of the mixture of gases to the purity threshold value, and indicating a purity threshold level of the hydrogen gas in the mixture of gases once the measured thermal conductivity of the mixture of gases reaches the purity threshold value.

In some embodiments, the mixture of gases is continuously provided from the vessel to the gas analyzer and the thermal conductivity of the mixture of gases is continuously measured by the gas analyzer.

In certain embodiments, the measured thermal conductivity of the mixture of gases is continuously compared to the purity threshold value until the measured thermal conductivity of the mixture of gases reaches the purity threshold value.

In some embodiments, the method also includes providing a pre-determined purge timeout time period, calculating a time period it takes for the measured thermal conductivity of the mixture of gases to reach the purity threshold value, and comparing the calculated time period to the pre-determined purge timeout period. In certain of these embodiments, a purge error is signaled if the calculated time period is longer than the pre-determined purge timeout period.

In some embodiments, the method further includes determining a volume of the vessel, determining a flow rate of the mixture of gasses from the vessel, and calculating the pre-determined purge timeout period based on the volume of the vessel and/or the flow rate.

The foregoing and other aspects, features, and advantages of the application will become more apparent from the following description and from the claims.

### BRIEF DESCRIPTION OF THE DRAWINGS

The features of the application can be better understood with reference to the drawings described below, and the claims. The drawings are not necessarily to scale, emphasis instead generally being placed upon illustrating the principles described herein. In the drawings, like numerals are used to indicate like parts throughout the various views.

FIG. 1 is a schematic illustration of the system for measuring a concentration of a reference gas according to one exemplary embodiment of the present invention.

FIG. 2 is a graph showing measurement of a concentration of a reference gas in a vessel.

FIG. 3 is a flow diagram illustrating the method of measuring a concentration of a reference gas according to an exemplary embodiment of the present invention.

### DETAILED DESCRIPTION OF THE INVENTION

It is noted that, as used in the specification and the claims, the singular form “a,” “an,” and “the” comprises plural referents unless the context clearly indicates otherwise. For example, reference to a component in the singular is intended to comprise a plurality of components.

The term “or” means “and/or” unless clearly indicated otherwise by context.

Reference throughout the specification to “an embodiment”, “another embodiment”, “some embodiments”, and so forth, means that a particular element (e.g., feature, structure, step, or characteristic) described in connection with the embodiment is included in at least one embodiment described herein, and may or may not be present in other embodiments. In addition, it is to be understood that the described elements may be combined in any suitable manner in the various embodiments.

“Optional” or “optionally” means that the subsequently described event or circumstance may or may not occur, and that the description includes instances where the event occurs and instances where it does not.

The terms “first,” “second,” and the like, “primary,” “secondary,” and the like, as used herein do not denote any order, quantity, or importance, but rather are used to distinguish one element from another.

The terms “front”, “back”, “bottom”, and/or “top” are used herein, unless otherwise noted, merely for convenience of description, and are not limited to any one position or spatial orientation.

The term “about” is to be construed as modifying a term or value such that it is not an absolute. This term will be defined by the circumstances. This includes, at the very least, the degree of expected experimental error, technique error and instrument error for a given technique used to measure a value. In general, this term used in connection with a numerical value throughout the specification and the claims denotes an interval of accuracy, familiar and acceptable to a person skilled in the art. In general, such interval of accuracy is ±10%. Thus, “about ten” means 9 to 11. All numbers in this description indicating amounts, ratios of materials, physical properties of materials, and/or use are to be understood as modified by the word “about,” except as otherwise explicitly indicated.

The term “comprising” is synonymous with “including,” “having,” “containing,” or “characterized by.” These terms are inclusive and open-ended and do not exclude additional, unrecited elements or method steps.

Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

Embodiments of the present invention disclosed herein are directed to a system and method for measuring an amount of a reference gas. The system and method of the present technology use the thermal conductivity of a pure gas sample as a reference and compares it to a contaminated gas sample on the analytical side of the gas detector. Once these two measured values are equal each other or within a particular margin, the sample gas on the analytical side is considered as pure as the reference gas. The present technology provides a simple measurement solution that is less expensive than other reference measurement technologies that look for specific impurities in the sample gas. The present system and method are designed to provide real-time continuous measurement and results and enable more accurate measurement than any reference thermal conductivity technology in this application.

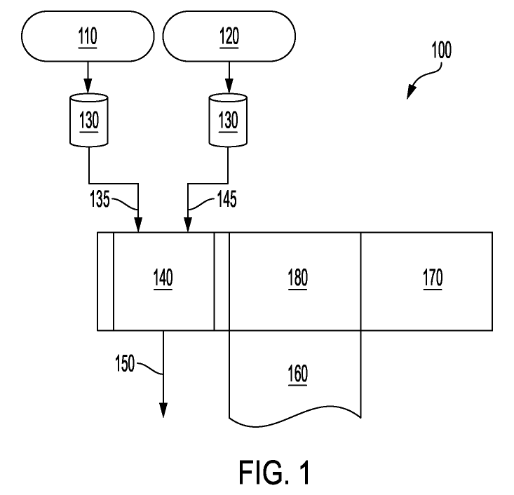

In some exemplary embodiments, such as shown in FIG. 1, the system 100 includes a source of sample gas 110 to be measured and a source of pure reference gas 120, both being fluidly coupled to a thermal conductivity detector 140 via a first conduit 135 and a second conduit 145, respectively. The first and second conduits 135, 145 may be connected to one or more flow meters 130 that measure the flow of gas therethrough and supply the measurement data to a flow control device. The conduits may also be provided with one or more restrictors for measuring and adjusting gas flow rates to the thermal conductivity detector. Suitable restrictors may have an adjustable conductance for changing/adjusting the gas flow and/or pressure. The adjustable conductance restrictors allow for selective adjustment of pressure and/or flow, for example manually or automatically, by a pressure control device.

The present technology may utilize any suitable thermal conductivity detector device 140. In one preferred embodiment, the present technology uses a micro thermal conductivity detector (Micro TCD), such as, for example, Micro GC Fusion™ device available from Inficon, Inc.

Micro TCD is a compact, sensitive and energy-efficient device used to measure the concentration of gases based on their thermal conductivity, making the system suitable for portable applications or environments with limited space. TCDs are used in gas chromatography (GC) systems to detect and quantify gases in a sample by comparing the thermal conductivity of the gas to a reference gas. The micro version is a miniaturized, low-power variant of the traditional TCD that may be integrated into portable devices or systems where space and energy efficiency are essential. A Micro TCD may include the following key components: thermistor/filament (sensing element), sample and reference gas flow channels, a bridge circuit, heat sink/insulating layer, micro heater control, and miniaturized housing.

The sensing element may be made of a resistive material such as platinum, tungsten, or other metal wires. Alternatively, a thermistor (a semiconductor whose resistance changes with temperature) may be used instead of a filament. The filament is heated electrically, and its resistance changes as it exchanges heat with the surrounding gas. The rate of heat loss depends on the thermal conductivity of the gas flowing over the filament.

In an exemplary embodiment of the present technology, the detector may have two gas flow channels: one for the sample gas and another for the reference gas. The reference gas channel and sample gas channel may be constructed from stainless steel for durability and resistance to corrosion, or from polymeric materials for lightweight and cost-effective applications. These channels may be placed in a close proximity to ensure that both the sample and reference gas experience similar environmental conditions. The sample gas channel is fluidly coupled to the sample gas source 110 through the first conduit 135, and the reference gas channel is fluidly coupled to the reference gas source 120 through the second conduit 145. In some embodiments, the second conduit 145 that supplies the reference gas is fluidly coupled to both the sample gas channel and the reference gas channel in the detector such that the reference gas may be supplied to both the sample gas and reference gas channels.

The sensing elements/filaments of the TCD may be integrated into a Wheatstone bridge configuration, which is an electrical circuit designed to measure small changes in resistance. Two filaments may be placed in the bridge: one in the sample gas flow channel and one in the reference gas flow channel. Changes in the resistance of the filaments due to the difference in thermal conductivity between the two gases cause an imbalance in the bridge, which generates a measurable signal. To minimize heat loss to the surroundings, the sensing elements may be thermally insulated or placed near heat sinks that help maintain the temperature within the detector. A precise micro-heater control mechanism may be included to maintain a stable filament temperature because fluctuations in the filament temperature can affect the detector’s sensitivity and accuracy.

A micro TCD may be enclosed in a compact housing that contains the micro-scale elements and gas flow channels. The size reduction allows for lower power consumption and faster thermal response times compared to traditional TCDs. The thermal conductivity detector 140 may be connected to an outlet or a vent 150 for evacuating the gases from the detector. The outlet 150 may be provided with a vent valve that may be selectively openable and closable for purging the gases from the detector 140 once the testing is completed.

The system further includes a controller coupled to the gas detector 140 for receiving and processing data from the detector and for monitoring and operation control of the system 100. Any suitable controller may be used in the present technology. In some exemplary embodiments, a programmable logic controller (PLC) may be used. The PLC may operate to manage and control the flow of gases (sample and reference) through the thermal conductivity detector 140 by adjusting valves or flow controllers 130 automatically based on sensor input. Further, the PLC may control the electrical current supplied to the filament heater to maintain a constant temperature, compensating for any fluctuations that could affect the detector’s performance. The resistance change in the thermal conductivity detector’s sensing element due to varying gas thermal conductivity results in a small electrical signal. The PLC processes these signals, compares them to reference values, and converts them into meaningful data such as gas concentration. The PLC may also be used to automate triggering data acquisition systems at specific time intervals, zero-point calibration, and periodic purging, streamlining the process and reducing human intervention.

In some exemplary embodiments of the present technology, the system may utilize Modbus communication protocol (Modbus API) to facilitate data exchange between the thermal conductivity detector 140 and other devices or systems, including PLCs, supervisory control systems, or data acquisition systems. Modbus API is an open, standardized protocol commonly used in industrial automation and control applications to transmit data between devices over serial lines or Ethernet networks. The Modbus interface allows the thermal conductivity detector 140 to transmit real-time data related to gas concentrations, temperature readings, flow rates, and other operational parameters. The Modbus API facilitates communication between the thermal conductivity detector 140 and external control systems, enabling data to be read from the thermal conductivity detector or commands to be written to it (e.g., starting or stopping the measurement process). The thermal conductivity detector 140 may use the Modbus API to communicate directly with the controller 160.

The present technology is particularly suitable for applications where a gas containing vessel needs to be purged with another gas, such as hydrogen. In these processes, it is important to ensure that the vessels are sufficiently flushed and contain pure or sufficiently pure purging gas after flushing. The present system and method provide an efficient, accurate and less expensive way for measuring whether the vessel has been sufficiently flushed with the purge gas, in particular hydrogen.

The present system 100 operates by first supplying a pure or substantially pure reference gas from the reference gas source 120. In one preferred embodiment, the reference gas is hydrogen. Hydrogen has the highest thermal conductivity of all gases and is particularly suitable for the application of the present technology. In other exemplary embodiments, the reference gas may be helium, argon, or other gases. The reference gas is supplied to both the reference and sample channels of the gas detector 140 (Micro TCD). The thermal conductivity of the pure reference gas is measured and recorded by the system 100. Other environmental conditions, such as temperature and gas flow/pressure are also measured and recorded by the system. The system then calibrates the gas detector 140 based on the measured conductivity of the reference gas and the measured environmental conditions.

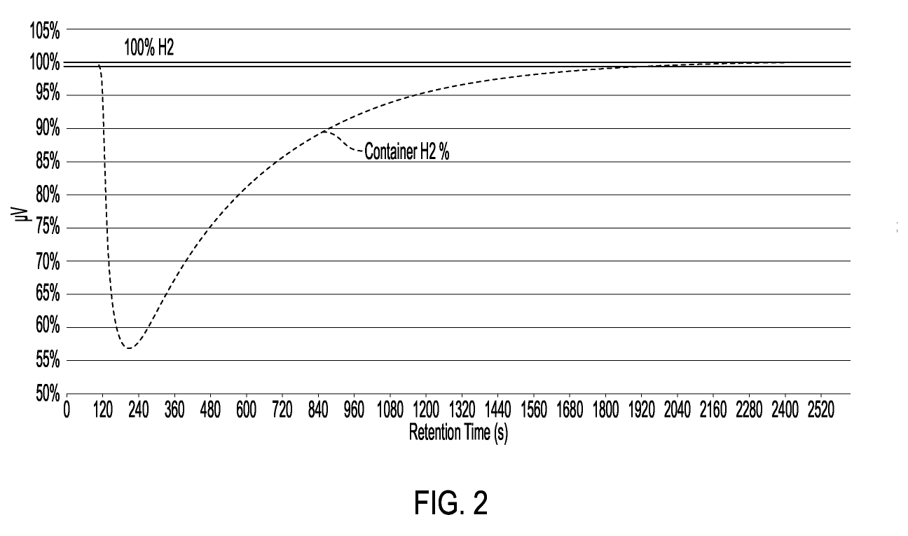

Next, a user switches to a vessel to be purged containing a different gas, such as nitrogen. The vessel is then gradually filled with a purge gas, such as hydrogen, and a sample gas from the vessel 110 is continuously supplied to the gas detector 140, which continuously measures and records the sample gas conductivity. As the vessel 110 is being purged, the conductivity of the sample gas is continuously changing until it reaches the value previously recorded for the reference gas. In case of hydrogen reference gas, the conductivity of the sample gas will increase as the vessel is being flushed with hydrogen, until it reaches a plateau near the zero point of the initial calibration, as shown in FIG. 2. Any variation in ambient temperature or pressure may be removed by the system 100 by using the previously measured data from the reference gas line.

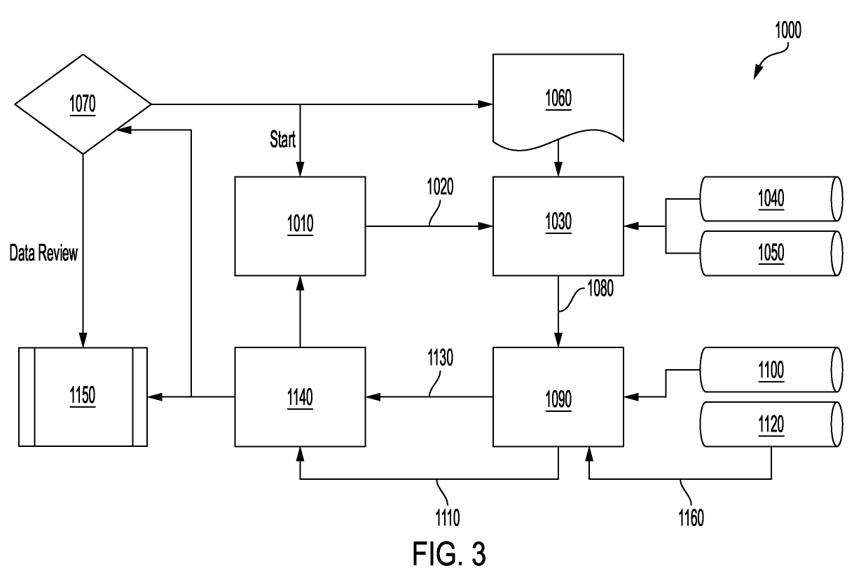

One exemplary method 1000 of the present invention is illustrated in FIG. 3. Initially, the system 100 is in an idle mode 1010, which indicates the state of the system while the instrument is not being used. Next, the user initiates a start command 1020 via a user interface, such as graphical user interface or application programming interface provided with the system 100, which instructs the system to initialize measuring 1030 the reference gas (hydrogen or another gas) with both the analytical and reference device channels. The reference gas is supplied to the thermal conductivity detector 140 and is floated through both reference channel 1050 and sample channel 1040 and a thermal conductivity of the reference gas is measured. The measured conductivity value is transmitted to the controller 160 and is used to calibrate/zero 1030 the detector 140 based on current environmental factors, such as temperature and pressure.

Custom configuration settings/parameters 1060 may be used to modify the measurement state. These parameters may be inputted 1070 via the user interface and may include a purity threshold, purge timeout and flow rate. In some embodiments, the user may select a setting based on the purity level required in the vessel. In some embodiments, the purity level may be around 90%, or around 95%, or around 99%, or around 99.5%, or around 100%. When this setting is selected with a particular purity level, the system will compare the continuously measured conductivity of the sample gas from the vessel to the previously measured conductivity of the pure reference gas and will signal when the conductivity of the sample gas is within the selected purity threshold range – e.g., within around 10%, or around 5%, or around 1%, or around 0.5% of the reference gas conductivity.

The system may also have a purge timeout user setting to determine an error fail state where the vessel did not reach a purity threshold value based on vessel size and process flow. The system may measure a vessel size and/or gas flow or these values may be manually inputted into the system 100 by the user. The system will then calculate a pre-determined time period during which the purge is expected to be completed.

Alternatively, the pre-determined time period may be inputted by the user. Once the sample gas is supplied 1100 to the system 100 and the conductivity is measured 1090 by the detector 140, the system will also measure a time period it takes for the conductivity of the sample gas to reach the selected purity threshold. If the measured time period passes the pre-determined time period (purge timeout) 1110, the system 100 may stop measurement and/or generate a failure signal indicating that the purge was not successful.

The system may further include a flow rate setting, which may be measured in cc/min. This setting is used to set the mass flow controllers inside the device to match the flow rates of the reference and analytical channels of the detector 140. Other parameters such as sample names and sample specific information may also be added to the system via the user interface.

The system may utilize the purity threshold parameter chosen by the user to calculate 1080 the threshold microvolt value, which is stored in the detector processing unit 180. Next, the system will move into measurement state 1090 to initialize measuring the sample gas. The sample gas is supplied 1120 to the thermal conductivity detector 140 from the sample gas vessel 110 and is floated through the sample channel. The detector 140 measures a thermal conductivity of the sample gas as the vessel starts to purge with the reference gas, e.g., hydrogen and the measured values are continuously supplied to the detector processing unit 180. As a concentration of the reference gas increases in the vessel 110, the sample gas thermal conductivity signal will increase until it reaches 1130 the microvolt purity threshold value stored in the system, indicating that the purity threshold limit of the reference gas in the sample vessel 110 has been reached. Once the purity threshold limit is reached, the system 100 may provide a summary 1140 of the measurement via the user interface and save the results to a database 1150.

In some embodiments, the detector 140 may continuously measure the thermal conductivity of the reference gas via the reference channel as the purge continues. The measurement data is supplied to the processor 180, which may be configured to continuously update 1160 the stored purity threshold limit to remove changing environment variables during the purge, such as temperature and pressure.

If the pre-determined purge timeout parameter is reached prior to reaching the purity threshold value, the system will stop the measurement process and a failure summary report may be generated and saved to the database. In some embodiments, the system may also generate a failure signal via the user interface 170, e.g., display, indicating that the purge was not successful. The system will return to the idle state 1010 and wait for another measurement start command.

While the present invention has been particularly shown and described with reference to certain exemplary embodiments, it will be understood by one skilled in the art that various changes in detail may be effected therein without departing from the spirit and scope of the invention that can be supported by the written description and drawings. Further, where exemplary embodiments are described with reference to a certain number of elements it will be understood that the exemplary embodiments can be practiced utilizing either less than or more than the certain number of elements.

The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A system for continuously monitoring and measuring a quantity of a reference gas, comprising:
a reference gas inlet fluidly coupled to a reference gas conduit,
a sample gas inlet fluidly coupled to a sample gas conduit,
a gas analyzer fluidly coupled to the reference gas conduit and the sample gas conduit and comprising a sample gas flow channel and a reference gas flow channel, wherein the reference gas conduit is fluidly coupled to both the reference gas flow channel and the sample gas flow channel, and
a controller connected to the gas analyzer,
wherein the gas analyzer is configured to measure a thermal conductivity of the reference gas and a thermal conductivity of a sample gas containing the reference gas and at least one other gas, and
wherein the controller is configured to calculate a purity threshold value based on the thermal conductivity of the reference gas and to compare the thermal conductivity of the sample gas to the purity threshold value to indicate a purity threshold level of the reference gas in the sample gas once the thermal conductivity of the sample gas reaches the purity threshold value.

2. The system according to claim 1, wherein the gas analyzer is a thermal conductivity detector.

3. The system according to claim 1, wherein the reference gas comprises hydrogen.

4. The system according to claim 1, further comprising:
a first valve positioned in the reference gas conduit and configured to control flow of the reference gas to the sample gas flow channel, and
a second valve positioned in the sample gas conduit and configured to control flow of the sample gas to the sample gas flow channel,
wherein the controller is configured to open the first valve to allow flow of the reference gas to the sample gas flow channel to calculate the purity threshold value, and to open the second valve to allow flow of the sample gas to the sample gas flow channel to measure the thermal conductivity of the sample gas.

5. The system according to claim 1, further comprising:
a first flow meter positioned in the reference gas conduit downstream from the reference gas inlet and configured to measure a flow rate of the reference gas, and
a second flow meter positioned in the sample gas conduit downstream from the sample gas inlet and configured to measure a flow rate of the sample gas.

6. The system according to claim 5, further comprising:
a first restrictor positioned in the reference gas conduit downstream from the first flow meter and configured to adjust at least one of the flow rate and a pressure of the reference gas, and
a second restrictor positioned in the sample gas conduit downstream from the second flow meter and configured to adjust at least one of the flow rate and a pressure of the sample gas.

7. The system of claim 6, wherein the controller is further configured to actuate at least one of the first restrictor and the second restrictor to maintain substantially equal flow rates and pressures in the reference gas flow channel and the sample gas flow channel.

8. The system of claim 1, further comprising a digital communication interface configured to transmit real-time purity values, dynamically updated purity thresholds, and system status data from the controller to an external supervisory device.

9. A method for continuously monitoring and measuring a quantity of a reference gas in a mixture of gases, comprising the steps of:
providing a reference gas and measuring a thermal conductivity of the reference gas via a gas analyzer,
calculating via a controller a purity threshold value based on the measured thermal conductivity of the reference gas,
providing the mixture of gases containing the reference gas and continuously measuring a thermal conductivity of the mixture of gases via the gas analyzer,
comparing the measured thermal conductivity of the mixture of gases to the purity threshold value via the controller, and
indicating a purity threshold level of the reference gas in the mixture of gases once the measured thermal conductivity of the mixture of gases reaches the purity threshold value.

10. The method according to claim 9, wherein the reference gas comprises hydrogen gas.

11. The method according to claim 9, further comprising the step of calibrating the gas analyzer based on the measured thermal conductivity of the reference gas.

12. The method according to claim 9, further comprising the steps of:
continuously providing the reference gas to the gas analyzer and continuously measuring the thermal conductivity of the reference gas, and
continuously updating the purity threshold value via the controller to remove changing environment variables.

13. The method according to claim 9, further comprising the steps of
measuring a flow rate of at least one of the reference gas and the mixture of gases, and
adjusting the flow rate of reference gas and the flow rate of the mixture of gases to be substantially equal.

14. A method for detecting a quantity of hydrogen gas during a purge of at least one other gas with the hydrogen gas, comprising the steps of:
providing a substantially pure hydrogen gas and measuring a thermal conductivity of the substantially pure hydrogen gas via a thermal conductivity detector,
calculating a purity threshold value via a controller coupled to the thermal conductivity detector based on the measured thermal conductivity of the substantially pure hydrogen gas,
supplying the substantially pure hydrogen gas to a vessel containing the at least one gas,
providing a mixture of gases from the vessel to the thermal conductivity detector and measuring a thermal conductivity of the mixture of gases,
comparing the measured thermal conductivity of the mixture of gases to the purity threshold value via the controller, and
indicating a purity threshold level of the hydrogen gas in the mixture of gases once the measured thermal conductivity of the mixture of gases reaches the purity threshold value.

15. The method according to claim 14, wherein the mixture of gases is continuously provided from the vessel to the thermal conductivity detector and the thermal conductivity of the mixture of gases is continuously measured by the thermal conductivity detector.

16. The method according to claim 14, wherein the measured thermal conductivity of the mixture of gases is continuously compared to the purity threshold value via the controller until the measured thermal conductivity of the mixture of gases reaches the purity threshold value.

17. The method according to claim 14, further comprising the steps of:
providing a pre-determined purge timeout time period,
calculating a time period it takes for the measured thermal conductivity of the mixture of gases to reach the purity threshold value, and
comparing the calculated time period to the pre-determined purge timeout period.

18. The method according to claim 17, further comprising the step of signaling a purge error if the calculated time period is longer than the pre-determined purge timeout period.

19. The method according to claim 17, further comprising the steps of:
determining a volume of the vessel,
determining a flow rate of the mixture of gasses from the vessel, and
calculating the pre-determined purge timeout period based on the volume of the vessel and/or the flow rate.
